(19) United States
(12) Patent Application Publication
(10) Pub. No.: US 2008/0154035 A1
(43) Pub. Date: Jun. 26, 2008

(54) COUPLING METHOD BETWEEN INTRAMOLECULAR CARBON IN UNSATURATED HYDROCARBON COMPOUND USING ORGANO-INDIUM COMPOUND AS COUPLING AGENT

(75) Inventors: Phil Ho LEE, Chuncheon-si (KR); Kooyeon LEE, Chuncheon-si (KR); Dong SEOMOON, Chuncheon-si (KR); Hyunseok KIM, Chuncheon-si (KR)

Correspondence Address: CANTOR COLBURN, LLP, 20 Church Street, 22nd Floor, Hartford, CT, 06103, US

(73) Assignee: PHIL HO LEE, Chuncheon-si (KR)

(21) Appl. No.: 11/778,944

(22) Filed: Jul. 17, 2007

(30) Foreign Application Priority Data
Dec. 20, 2006 (KR) 10-2006-0130535

(51) Int. Cl. C07C 1/00; C07C 253/30; C07C 315/04; C07C 67/00; C07C 69/76; C07D 209/10; C07D 267/04; C07D 267/22

(52) U.S. Cl. 540/468; 540/552; 548/508; 558/431; 560/102; 560/76; 560/96; 568/34; 585/410

(57) ABSTRACT

The present invention relates to a coupling method between intramolecular carbon using the intramolecular cyclization reaction in situ of an allyl-indium compound derived from an allyl derivative containing an unsaturated hydrocarbon compound and indium, in presence of a transition metal compound catalyst. More particularly, the present invention relates to a coupling method between intramolecular carbon for preparing a cyclic compound having a vinyl group as a substituent by bonding a carbon in an allyl derivative containing an unsaturated hydrocarbon compound and a carbon in the unsaturated hydrocarbon compound via intramolecular cyclization reaction in situ of an allyl-indium compound as a coupling agent derived from the allyl derivative containing the unsaturated hydrocarbon and indium (In), in presence of a palladium catalyst.

### CROSS-REFERENCE TO RELATED APPLICATIONS

This application claims priority to Korean Patent Application No. 10-2006-0130535, filed on Dec. 20, 2006, in the Korean Intellectual Property Office, and all the benefits accruing therefrom under 35 U.S.C. §119, the disclosure of which is incorporated herein in its entirety by reference.

### BACKGROUND OF THE INVENTION

1. Field of the Invention

The present invention relates to a coupling method between intramolecular carbon using the intramolecular cyclization reaction in situ of an allyl-indium compound derived from an allyl derivative containing an unsaturated hydrocarbon compound and indium, in presence of a transition metal compound catalyst. More particularly, the present invention relates to a coupling method between intramolecular carbon for preparing a cyclic compound having a vinyl group as a substituent by bonding a carbon in an allyl derivative containing an unsaturated hydrocarbon compound and a carbon in the unsaturated hydrocarbon compound via intramolecular cyclization reaction in situ of an allyl-indium compound as a coupling agent derived from the allyl derivative containing the unsaturated hydrocarbon and indium (In), in presence of a palladium catalyst.

2. Description of the Related Art

Examples of the allylation methods known up to this point include the Friedel-Crafts alkylation (Org. React. 1946, 3, 1), Claisen Rearrangement (Chem. Ber. 1912, 45, 3157), Organometallic reagent and substitution reaction of allyl halide (Tetrahedron Lett. 1990, 31. 4413), Palladium-catalyzed allyl cross-coupling reaction (Pure Appl. Chem. 1985, 57, 1771), and the like. Among these, the palladium-catalyzed allyl cross-coupling reaction is known to be the most effective allylation method.

The most well known example of the allyl cross-coupling reaction is the Stille cross-coupling reaction which uses an allyl tin (Sn) compound as a coupling agent. The allyl tin (Sn) compound is stable in water and air, and has good selectivity to various functional groups. Thus, the allyl tin (Sn) compound has been in the limelight.

In general, the allyl tin (Sn) compound is easily prepared. However, the reaction progress is occasionally unsuitable, and it is difficult to prepare a desired allyl metal. Moreover, allyl halides are used for the preparation of allyl metal reagent. Among the allyl halides, allyl bromide and allyl iodide are mainly used, while allyl chloride is used as a form of iodide salt. Moreover, the tin compound has toxicity.

Therefore, in order to solve these problems, an in situ method has been used. The in situ method uses the product, obtained by reacting a metal and an allyl halide, directly in the coupling reaction without carrying out an additional purification process to separate allyl anions from the product. For example, organomagnesium and organolithium compounds are used widely as a coupling agent, because they may be used in the coupling reaction without an additional purification process of allyl anions obtained by reacting a metal and an allyl halide.

However, the above-mentioned coupling agents also have strong reactivity to various functional groups in the substrates. Thus, it is disadvantageous in that selection range of a functional group in the coupling reaction is narrow.

Therefore, a method for forming a carbon-carbon bond between an unsaturated hydrocarbon compound and an allyl derivative using an organometallic compound is demanded, in which the organomatallic compound has no toxicity and has wide selection range of functional groups in addition to being usable in the coupling reaction without carrying out an additional purification reaction.

### SUMMARY OF THE INVENTION

Therefore, the present invention has been made in view of the above problems, and it is an object of the present invention to provide a coupling method between intramolecular carbon which introduces a vinyl group to an unsaturated hydrocarbon compound by the in situ cyclization reaction of an allyl-indium compound derived from indium having low toxicity and an allyl derivative containing the unsaturated hydrocarbon compound, in presence of a transition metal compound catalyst.

In accordance with the present invention, the above and other objects can be accomplished by the provision of a coupling method between intramolecular carbon which introduces a vinyl group to an unsaturated hydrocarbon compound by in situ reacting an allyl-indium compound derived from an allyl derivative containing the unsaturated hydrocarbon compound with indium.

### DESCRIPTION OF THE PREFERRED EMBODIMENTS

The present invention will now be described in greater detail.

The present invention relates to a coupling method between intramolecular carbon for preparing a cyclic compound represented by the following chemical formula 1 or 2 having a vinyl group as a substituent by in situ reacting an allyl derivative represented by the following chemical formula 3 or 4 containing an unsaturated hydrocarbon compound with indium (In) or an indium halide and a tertiary anime represented by the following chemical formula 5 to form an allyl-indium compound in which Z of the allyl derivative represented by the following chemical formula 3 or 4 is substituted with indium, and carrying out the intramolecular the in situ cyclization reaction.

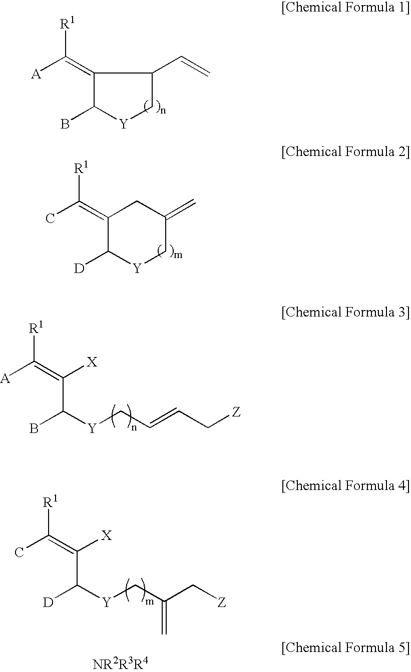

wherein, X is Cl, Br or I; Y is (CH₂)ₚCR¹¹R¹², NR¹³ or (CH₂)_{q}ONR¹⁴; Z is Cl, Br, I, OAc or OCO₂CH₃; A and B are each independently hydrogen, alkyl having 1 to 5 carbon atoms, phenyl or benzyl, or may form an alkyl ring or fused ring by being bonded to alkylene having 2 to 10 carbon atoms or alkylene having 2 to 10 carbon atoms containing a fused ring; R¹ is each independently hydrogen, fluoro-substituted or -unsubstituted alkyl having 1 to 5 carbon atoms, alkoxy having 1 to 5 carbon atoms, alkylcarbonyl having 1 to 5 carbon atoms or alkoxycarbonyl having 1 to 5 carbon atoms; R², R³ and R⁴ are each independently alkyl having 1 to 5 carbon atoms, phenyl or benzyl; R¹¹and R¹² are each independently hydrogen alkyl having 1 to 5 carbon atoms, alkoxy having 1 to 5 carbon atoms, alkylcarbonyl having 1 to 5 carbon atoms or alkoxycarbonyl having 1 to 5 carbon atoms; R¹³ is alkyl having 1 to 5 carbon atoms or SO₂R²¹; R¹⁴ is t-butoxycarbonyl or alkyl having 1 to 5 carbon atoms; R²¹ is alkyl having 1 to 5 carbon atoms or phenyl; n is an integer of 1 to 4; m is an integer of 1 to 3; p is an integer of 0 to 5; and q is an integer of 1 to 3.

When Z of the allyl derivative represented by chemical formula 2 or 3 is a halogen, i.e. Cl, Br or I, the allyl derivative and indium (In) are reacted in situ. When Z is OAc or OCO₂CH₃, the allyl derivative, indium (In), an indium halide, and a tertiary amine represented by the chemical formula 5 are reacted in situ.

Furthermore, a method for preparing a cyclic compound having a vinyl group represented by the chemical formula 1 or 2 as a substituent is provided, comprising forming an allyl-indium compound in which Z of the allyl derivative represented by the chemical formula 2 or 3 is substituted with indium by the above reaction, and carrying out the intramolecular cyclization reaction in situ.

Specific examples of the allyl derivative represented by the chemical formula 3 include compounds represented by the following chemical formulas 6 to 8. However, the present invention is not limited thereto.

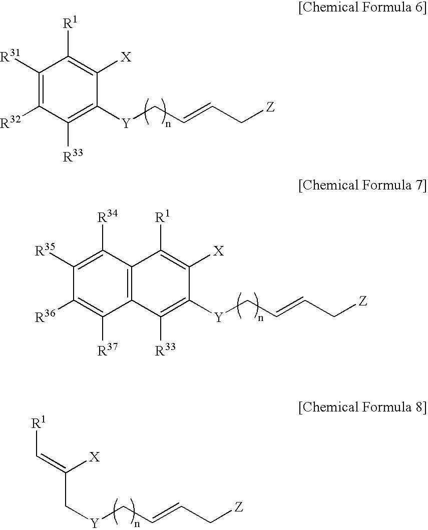

wherein, R¹, X, Y, Z and n are the same as defined in the chemical formula 3, and R³¹ to R³⁷ are each independently hydrogen, alkyl having 1 to 5 carbon atoms, alkoxy having 1 to 5 carbon atoms, alkylcarbonyl having 1 to 5 carbon atoms or alkoxycarbonyl having 1 to 5 carbon atoms.

Specific examples of the allyl derivative represented by the chemical formula 4 include compounds represented by the following chemical formulas 9 to 10. However, the present invention is not limited thereto.

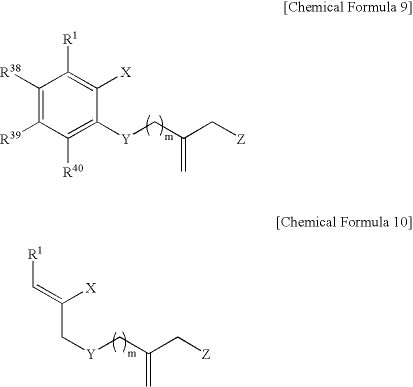

wherein, R¹, X, Y, Z and m are the same as defined in the chemical formula 4, and R³⁸ to R⁴⁰ are each independently hydrogen, alkyl having 1 to 5 carbon atoms, alkoxy having 1 to 5 carbon atoms, alkylcarbonyl having 1 to 5 carbon atoms or alkoxycarbonyl having 1 to 5 carbon atoms.

Examples of the cyclic compound substituted with the vinyl group represented by the chemical formula 1 or 2 prepared by the coupling method between intramolecular carbon according to the present invention include the following compounds.

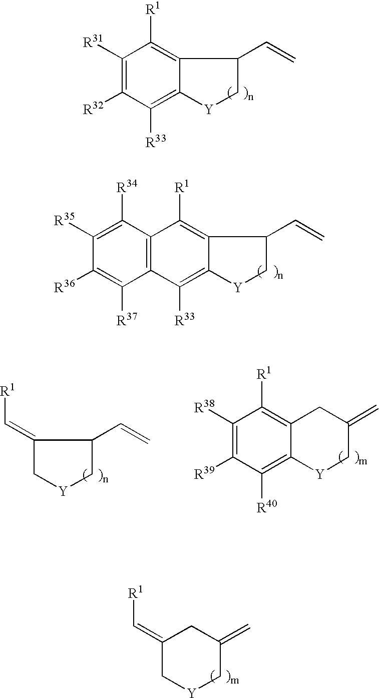

In the present invention, “in situ method” means that allyl-indium compound, in which Z of the allyl derivative represented by the chemical formula 3 or 4 produced by reacting the allyl derivative represented by the chemical formula 3 or 4 with indium (In) or an indium halide and a tertiary amine represented by the following chemical formula 5 substituted with indium, is used in the reaction as it is without carrying out an additional purification process.

When Z in the chemical formula 3 or 4 is Cl, Br or I, indium is used alone in the reaction. When Z is OAc or OCO₂CH₃, indium (In) and an indium halide being used in combination instead of alone may reduce the reaction time and obtain a cyclic compound with improved yield. The content of the indium and indium halide is preferably 1 to 3 equivalent or 0.1 to 1.0 equivalent based on the compound represented by chemical formula 3 or 4, respectively.

As the transition metal catalyst used in the present invention, a palladium catalyst is used. Preferably, the palladium catalyst is selected from the group consisting of PdCl₂, Pd(OAc)₂, Pd(CH₃CN)₂Cl₂, Pd(PhCN)₂Cl₂, Pd₂dba₃CHCl₃ and Pd(PPh₃)₄, and more preferably from Pd₂dba₃CHCl₃ and Pd(PPh₃)₄. The content of the palladium catalyst, in terms of catalytic amount, is preferably 4 to 10 mol %. When the content is less than 4 mol %, the yield is reduced.

Moreover, when Z in the chemical formula 3 or 4 is Cl, Br or I, an organic base is not used. On the other hand, when Z is OAc or OCO₂CH₃, a tertiary amine represented by the chemical formula 5 is used as an organic base. The tertiary amine functions to increase the yield of the desired cyclic compound in the present invention by reducing the production of unreacted compounds. R², R³ and R⁴ of the tertiary amine are preferably selected from each independently methyl, ethyl or butyl, and it is the most preferable to use n-butyldimethylamine (n-BuNMe₂). It is preferable to use 2 to 5 equivalent of the tertiary amine based on the compound represented by the chemical formula 3 or 4. Meanwhile, the yield is low when an inorganic base such as potassium carbonate is used.

The in situ cyclization reaction is carried out in presence of a dimethylformamide (DMF) or tetrahydrofuran (THF) solvent, and at a reaction temperature in the range of 90 to 110° C.

Furthermore, a lithium halide may be used as an additive to progress the reaction efficiently. It is preferable to use lithium chloride (LiCl) to improve the cyclic compound yield while reducing the reaction time. And, it is preferable to use the lithium chloride in a content of 1.0 to 4.0 equivalent based on the compound represented by the chemical formula 2.

Hereinafter, the present invention will be explained in more detail with reference to the following examples. However, these examples are given for the purpose of illustration and are not to be construed as limiting the scope of the invention.

### EXAMPLES

### Example 1

### Preparation of diethyl 2,3-dihydro-3-vinylindene-1,1-dicarboxylate

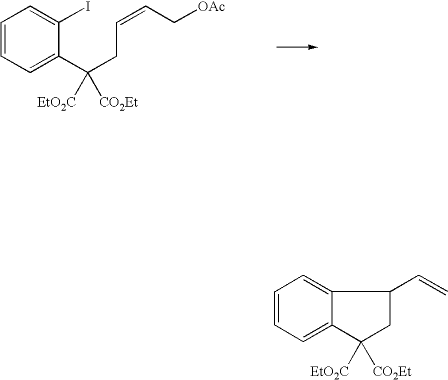

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and diethyl 2-(4-acetoxy-2-butenyl)-2-(2-iodophenyl)malonate (237.0 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 2,3-dihydro-3-vinylindene-1,1-dicarboxylate (115.1 mg, 80%).

¹H NMR (400 MHz, CDCl₃, 25° C., TMS): d=7.58 (d, J=6.96 Hz, 1H), 7.32-7.26 (m, 3H), (d, J=7.07 Hz, 1H), 5.80 (ddd, J=17.46, 9.59, 8.45 Hz, 1H), 5.23 (d, J=17.08 Hz, 1H), 5.17 (d, J=10.02 Hz, 1H), 4.26 (q, J=7.08 Hz, 2H), 4.22-4.13 (m, 2H), 3.93 (q, J=8.30 Hz, 1H), 3.04 (dd, J=13.29, 5.63 Hz, 1H), 2.39 (dd, J=13.31, 4.55 Hz, 1H), 1.30 (t, J=7.10, 3H), 1.24 (t, J=7.17 Hz, 3H)

### Example 2

### Preparation of ethyl 2,3-dihydro-3-vinyl-1H-indene-1-carboxylate

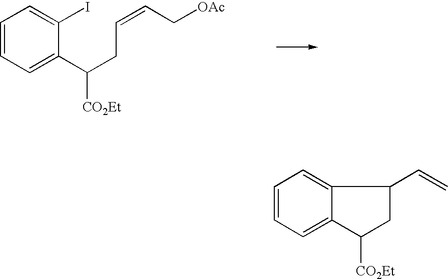

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 2-(4-acetoxy-2-butenyl)-2-(2-iodophenyl)ethyl acetate (201.0 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, ethyl 2,3-dihydro-3-vinyl-1H-indene-1-carboxylate (77.9 mg, 72%, isomer A: isomer B=1:1.2).

Isomer A; ¹H NMR (400 MHz, CDCl₃, 25° C., TMS): d=7.39 (d, J=7.10 Hz, 1H), 7.26-7.21 (m, 2H), 5.85 (ddd, J=8.84, 8.90, 17.26 Hz, 1H), 5.24-5.09 (m, 2H), 4.28-4.20 (m, 2H), 4.07-4.00 (m, 2H), 2.58 (ddd, J=7.76, 7.76, 12.87, Hz, 1H), 2.23 (ddd, J=9.46, 9.46, 12.86 Hz, 1H), 1.32 (t, J=7.11 Hz, 3H);

Isomer B ; ¹H NMR (400 MHz, CDCl₃, 25° C., TMS): d=7.39 (d, J=7.10 Hz, 1H), 7.26-7.21 (m, 2H), 5.85 (ddd, J=8.84, 8.90, 17.26 Hz, 1H), 5.24-5.09 (m, 2H), 4.15 (q, J=7.17 Hz, 2H), 4.07-4.00 (m, 1H), 3.74 (q, J=8.50 Hz, 1H), 2.71 (ddd, J=12.94, 7.89, 3.68 Hz, 1H), 2.10 (ddd, J=8.18, 8.18, 13.05 Hz, 1H), 1.26 (t, J=7.10 Hz, 3H)

### Example 3

### Preparation of 2,3-dihydro-1-vinyl-1H-indene

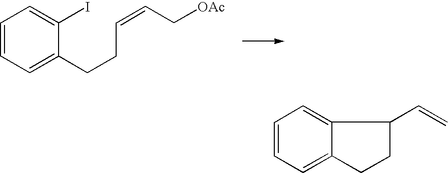

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 5-(2-iodophenyl)-pent-2-enyl acetate (165.0 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 2,3-dihydro-1-vinyl-1H-indene (50.5 mg, 70%).

¹H NMR (400 MHz, CDCl₃, 25° C., TMS): d=7.23-7.15 (m, 4 Hz), 5.86 (ddd, J=17.07, 9.99, 8.22 Hz, 1H), 5.18-5.07 (m, 2H), 3.75 (q, J=8.06 Hz, 1H), 2.99-2.84 (m, 2H), 2.40-2.28 (m, 1H), 2.15-2.04 (m, 1H)

### Example 4

### Preparation of 1-(p-toluenesulfonyl)-3-vinylindoline

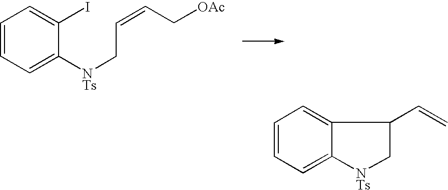

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 2-iodo-N-(4-acetoxy-2-butenyl)-N-tosylbenzenamine (242.7 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 1-(p-toluenesulfonyl)-3-vinylindoline (83.8 mg, 56%).

¹H NMR (400 MHz, CDCl₃, 25° C., TMS): d 7.66 (t, J=8.71 Hz, 3H), 7.22 (d, J=8.03 Hz, 3H), 7.01 (d, J=4.24 Hz, 2H), 5.53 (q, J=8.82 Hz, 1H), 5.08 (dt, J=11.62, 9.89 Hz, 2H), 4.14 (t, J=9.86 Hz, 1H), 3.73 (q, J=8.46 Hz, 1H), 3.59 (dd, J=10.68, 7.87 Hz, 1H), 2.37 (s, 3H)

### Example 5

### Preparation of diethyl 3,4-dihydro-4-vinylnaphthalene-2,2(1H)-dicarboxylate

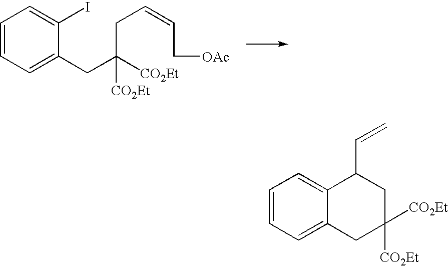

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 2-(4-acetoxy-2-butenyl)-2-(2-iodobenzyl)malonic acid diethyl ester (244.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 3,4-dihydro-4-vinylnaphthalene-2,2(1H)-dicarboxylate (93.7 mg, 62%).

¹H NMR (400 MHz, CDCl₃, 25° C., TMS): d=7.18-7.13 (m, 4H), 5.77 (ddd, J=17.06, 9.78, 9.30 Hz, 1H), 5.18 (dd, J=16.89, 9.86 Hz, 2H), 4.20 (qq, J=7.14, 7.11 Hz, 4H), 3.56-3.49 (m, 1H), 3.42-3.38 (m, 1H), 3.16 (d, J=16.30 Hz, 1H), 2.59 (ddd, J=13.50, 6.06, 2.10 Hz, 1H), 2.01 (dd, J=13.50, 11.20 Hz, 1H), 1.27 (t, J=7.14 Hz, 3H), 1.17 (t, J=7.08 Hz, 3H)

### Example 6

### Preparation of methyl 1,2,3,4-tetrahydro-2-(phenylsulfonyl)-4-vinylnaphthalene-2-carboxylate

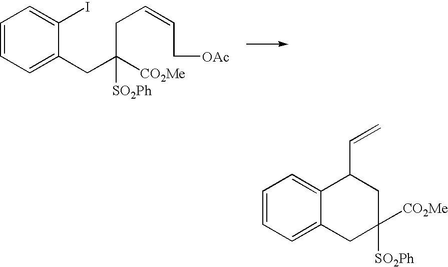

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 2-(4-acetoxy-2-butenyl)-2-benzenesulfonyl-2-(2-iodobenzyl)methyl acetate (271.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, methyl 1,2,3,4-tetrahydro-2-(phenylsulfonyl)-4-vinylnaphthalene-2-carboxylate (131.9 mg, 74%).

¹H NMR (400 MHz, CDCl₃, 25° C., TMS): d=7.86 (d, J 8.20 Hz, 2H), 7.73 (t, J=7.11 Hz, 1H), 7.60 (t, J=7.66 Hz, 2H), 7.15-7.10 (m, 4H), 5.72 (dt, J=26.24, 17.35 Hz, 1H), 5.22 (dd, J=17.05, 11.98 Hz, 2H), 3.59 (s, 3H), 3.56-3.40 (m, 4H), 2.67 (ddd, J=12.9, 6.05, 2.37 Hz, 1H), 2.09 (t, J=12.32 Hz, 1H)

### Example 7

### Preparation of 3,4-dihydro-4-vinylnaphthalene-2,2(1H)-dicarbonitrile

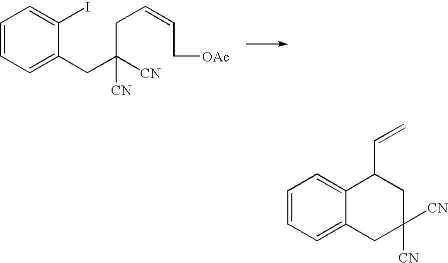

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 5,5-dicyano-(2-iodophenyl)-2-hexenyl acetate (197.1 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 3,4-dihydro4-vinylnaphthalene-2,2(1H)-dicarbonitrile (66.6 mg, 64%).

¹H NMR (400 MHz, CDCl₃, 25° C., TMS): d=7.28-7.23 (m, 4H), 7.13 (d, J=7.31 Hz, 1H), 5.75 (ddd, J=17.04, 9.23, 7.66 Hz, 1H), 5.37 (dd, J=14.47, 10.01 Hz, 2H), 3.86-3.79 (m, 1H), 3.49 (dt, J=23.79, 7.52 Hz, 3H), 2.68 (ddd, J=11.47, 6.02, 1.96 Hz, 1H), 2.19 (dd, J=13.32, 11.52 Hz, 1H)

### Example 8

### Preparation of 1,2,3,4-tetrahydro-3,3-bis(phenylsulfonyl)-1-vinylnaphthalene

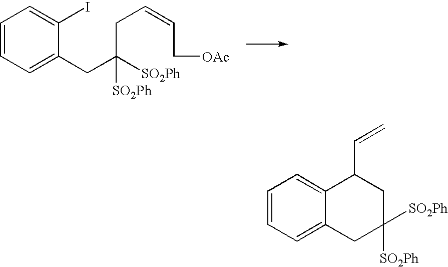

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 5,5-bis(phenylsulfonyl)-(2-iodophenyl)-2-hexenyl acetate (312.3 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 1,2,3,4-tetrahydro-3,3-bis(phenylsulfonyl)-1-vinylnaphthalene (157.9 mg, 72%).

¹H NMR (400 MHz, CDCl₃, 25° C., TMS): d=8.07 (d, J=8.15 Hz, 2H), 7.95 (d, J=8.18 Hz, 2H), 7.73 (t, J=7.35 Hz, 1H), 7.67-7.58 (m, 3H), 7.51 (t, J=7.75 Hz, 2H), 7.13 (t, J=7.27 Hz, 1H), 7.09 (t, J=6.42 Hz, 1H), 6.94 (d, J=7.28 Hz, 1H), 5.8 (dt, J=23.67, 16.96 Hz, 1H), 5.28 (d, j=10.12 Hz, 1H), 5.19 (d, J=17.04 Hz, 1H), 3.51 (d, J=7.57 Hz, 1H), 3.47-3.42 (m, 1H), 2.78 (dd, J=15.20, 4.74 Hz, 1H), 2.27 (dd, J=15.17, 11.82 Hz, 1H)

### Example 9

### Preparation of tert-butyl 4,5-dihydro-5-vinylbenzo[e][1,2]oxazepine-3(1H)-carboxylate

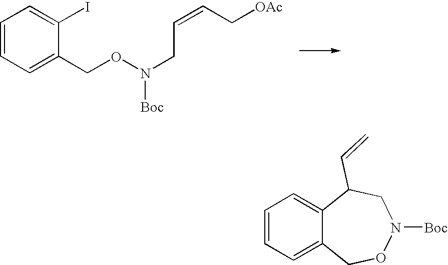

Under nitrogen atmosphere, indium (114.8 mg, 1.0 mmol), indium (III) chloride (55.29 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and 4-(2-iodobenxyloxy-t-butoxy-carbonylamino)-2-butenyl acetate (230.6 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, tert-butyl 4,5-dihydro-5-vinylbenzo[e][1,2]oxazepine-3(1H)-carboxylate (55.1 mg, 40%).

¹H NMR (400 MHz, CDCl₃, 25° C., TMS): d=7.29-7.20 (m, 4H), 6.15 (ddd, J=22.79, 10.41, 5.76 Hz, 1H), 5.17 (dt, J=14.02, 5.20 Hz, 2H), 5.00 (d, J=13.99 Hz, 1H), 4.86 (d, J=17.26 Hz, 1H), 4.19 (dd, J=13.56, 5.85 Hz, 1H), 3.82 (d, J=5.53 Hz, 1H), 3.76 (dd, J=13.57, 5.85 Hz, 1H), 1.48 (s, 9H)

### Example 10

### Preparation of diethyl 2,3-dihydro-5-methyl-3-vinylindene-1,1-dicarboxylate

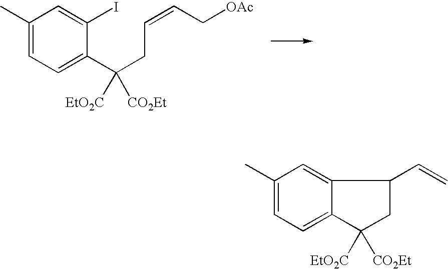

Under nitrogen atmosphere, indium (114.8 mg, 1.0 mmol), indium (III) chloride (55.3 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and diethyl 2-(4-acetoxy-2-butenyl)-2-(2-iodo-4-methylphenyl)malonate (244.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, diethyl 2,3-dihydro-5-methyl-3-vinylindene-l,1-dicarboxylate (120.9 mg, 80%).

¹H NMR (400 MHz, CDCl₃) d 7.09-7.05 (m, 1H), 7.01-6.94 (m, 2H), 6.24 (ddd, J=17.02, 9.67, 9.21 Hz, 1H), 4.92-4.99 (m, 2H), 4.16 (q, J=7.13 Hz, 4H), 3.59 (q, J=7.04 Hz, 1H), 2.87 (dd, J=13.42, 6.17 Hz, 1H), 2.62 (dd, J=13.43, 11.77 Hz, 1H), 2.35 (s, 3H), 1.31 (t, J=7.13 Hz, 6H)

### Example 11

### Preparation of diethyl 3,4-dihydro-7-methoxy-4-vinylnaphthalene-2,2(1H)-dicarboxylate

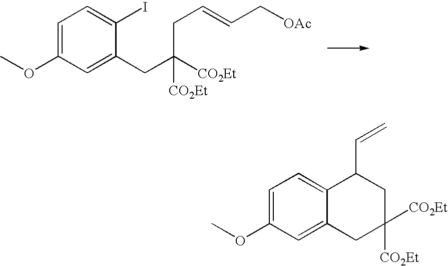

Under nitrogen atmosphere, indium (114.8 mg, 1.0 mmol), indium (III) chloride (55.3 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and diethyl 2-(2-iodo-5-methoxybenzyl)-2-(4-acetoxy-2-butenyl)malonate (259.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, diethyl 3,4-dihydro-7-methoxy-4-vinylnaphthalene-2,2(1H)-dicarboxylate (129.6 mg, 78%).

¹H NMR (400 MHz, CDCl₃) d 6.90 (d, J=7.54 Hz, 1H), 6.52 s, 1H), 6.44 (d, J=7.54 Hz, 1H), 5.79 (ddd, J=17.02, 9.67, 9.21 Hz, 1H), 5.07 (dd, J=16.77, 9.67 Hz, 2H), 4.20 (q, J=7.14 Hz, 4H), 3.73 (s, 3H), 3.56-3.49 (m, 1H), 3.42-3.38 (m, 1H), 3.16 (d, J=16.30 Hz, 1H), 2.59 (ddd, J=13.50, 6.06, 2.10 Hz, 1H), 2.01 (dd, J=13.50, 11.20 Hz, 1H), 1.29 (t, J=7.14 Hz, 6H)

### Example 12

### Preparation of 2,2-diethyl 7-methyl 3,4-dihydro-4-vinylnaphthalene-2,2,7(1H)-tricarboxylate

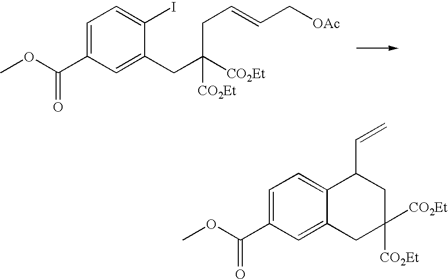

Under nitrogen atmosphere, indium (114.8 mg, 1.0 mmol), indium (III) chloride (55.3 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and methyl 3-(2,2-di(ethoxycarbonyl)-6-acetoxy-4-hexenyl)-4-iodobenzoate (273.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, 7-methyl 3,4-dihydro-4-vinylnaphthalene-2,2,7(1H)-tricarboxylate (142.4 mg, 79%).

¹H NMR (400 MHz, CDCl₃) d 7.72 (s, 1H), 7.64 (d, J=7.55 Hz, 1H), 7.12 (d, J=7.55 Hz, 1H), 6.01 (ddd, J=17.07, 9.69, 9.23 Hz, 1H), 4.93-4.98 (m, 2H), 4.12 (q, J=7.13 Hz, 4H), 3.88 (s, 3H), 3.53-3.56 (m, 3H), 2.59 (ddd, J=13.54, 6.07, 2.15 Hz, 1H), 2.01 (dd, J=13.54, 11.26 Hz, 1H), 1.30 (t, J=7.14 Hz, 6H)

### Example 13

### Preparation of diethyl 3,4-dihydro-4-vinylanthracene-2,2(1H)-dicarboxylate

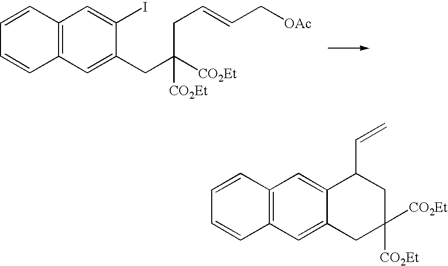

Under nitrogen atmosphere, indium (114.8 mg, 1.0 mmol), indium (III) chloride (55.3 mg, 0.25 mmol), lithium chloride (63.5 mg) and Pd(PPh₃)₄ (57.3 mg, 10 mol %) were mixed, and DMF (1 ml) was added to the mixture. n-butyldimethylamine (101.2 mg, 2.0 mmol) and diethyl 2-(4-acetoxy-2-butenyl)-2-((2-iodo-3-naphthalenyl)methyl)malonate (269.2 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, diethyl 3,4-dihydro-4-vinylanthracene-2,2(1H)-dicarboxylate (142.7 mg, 81%).

¹H NMR (400 MHz, CDCl₃) d 7.60-7.69 (m, 2H), 7.40 (s, 2H), 7.23-7.31 (m, 2H), 6.30 (ddd, J=17.07, 9.69, 9.23 Hz, 1H), 4.91-4.98 (m, 2H), 4.15 (q, J=7.13 Hz, 4H), 3.55-3.64 (m, 3H), 2.42 (dd, J=13.52, 6.07 Hz, 1H), 2.17 (dd, J=13.54, 11.37 Hz, 1H), 1.30 (t, J=7.14 Hz, 6H)

### Example 14

### Preparation of diethyl 3-methylene-4-vinylcyclopentane-1,1-dicarboxylate

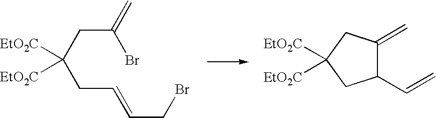

Under nitrogen atmosphere, indium (57.0 mg, 0.5 mmol), lithium chloride (63.5 mg) and Pd(PPh₃)₄ (23.1 mg, 4 mol %) were mixed, and DMF (1 ml) was added to the mixture. Diethyl 2-(2-bromoallyl)-2-(4-bromo-2-butenyl)malonate (206.1 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, diethyl 3-methylene-4-vinylcyclopentane-1,1-dicarboxylate (103.4 mg, 82%).

¹H NMR (400 MHz, CDCl₃) d 5.09-5.05 (m, 2H), 4.98 (d, J=2.20 Hz, 1H), 4.82 (d, J=2.20 Hz, 1H), 4.20 (q, J=7.18 Hz, 2H), 4.19 (q, J=7.18 Hz, 2H), 3.20-3.14 (m, 1H), 3.07 (d, J=17.5 Hz, 1H), 2.94 (dq, J=17.5, 2.30 Hz, 1H), 2.57 (dd, J=13.0, 7.65 Hz, 1H), 2.00 (dd, J=13.0, 10.9 Hz, 1H), 1.26 (t, J=7.08 Hz, 3H), 1.25 (t, J=7.08 Hz, 3H)

### Example 15

### Preparation of tert-butyl 5,6-dihydro-5-methylene-1H-benzo[f][1,2]oxazocine-3(4H)-carboxylate

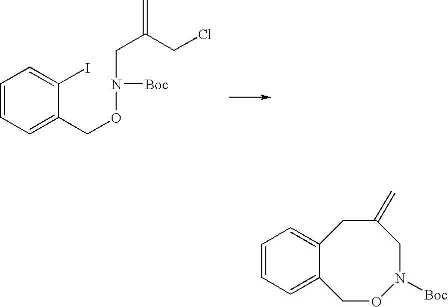

Under nitrogen atmosphere, indium (57.0 mg, 0.5 mmol), lithium chloride (63.5 mg) and Pd(PPh₃)₄ (23.1 mg, 4 mol %) were mixed, and DMF (1 ml) was added to the mixture. 3-(2-iodobenzyloxy-t-butoxycarbonylamino)-2-(chloromethyl)-1-propene (218.9 mg, 0.5 mmol) dissolved in DMF (1 ml) was added to the above reaction solution. While stirring for 1 hour at 100° C., the reaction was confirmed by TLC and GC. Then, the reaction was terminated by adding a saturated aqueous Na₂S₂O₃ solution (1 ml). The aqueous layer was extracted with Et₂O (20 ml×3) and washed with a saturated aqueous solution (20 ml) and a saturated aqueous NaCl solution (20 ml). The extracted organic layer was dried over anhydrous MgSO₄, and the residue was filtered off. After removing the solvent from the dried organic layer, column chromatography was carried out to isolate the title compound, tert-butyl 5,6-dihydro-5-methylene-1H-benzo[f][1,2]oxazocine-3(4H)-carboxylate (104.6 mg, 76%).

¹H NMR (400 MHz, CDCl₃) d 7.31-7.21 (m, 4H), 5.06 (s, 1H), 5.02 (s, 2H), 4.85 (s, 1H), 4.03 (s, 2H), 3.57 (s, 2H), 1.47 (s, 9H)

According to the present invention, the carbon-carbon coupling method by the in situ cyclization reaction in the unsaturated hydrocarbon compound using indium is capable of obtaining a cyclic compound, which coupled a carbon and a carbon in the unsaturated compound by the in situ reaction without carrying out an additional purification process to isolate the intermediate compound produced by the transition metal catalyst and indium, with high yield.

Since the intramolecular carbon-carbon coupling reaction is widely used in synthesizing natural substances, medicines and agrichemicals, the coupling method of carbon-carbon in the unsaturated carbon compound using indium can be used in synthesizing polyolefin macrolide, rapamycin, verginiamycin, strychnine, papuamine, heliclonadiamine, goniofurfurone, and the like. The coupling method is usable in obtaining a cyclic compound which is carbon-carbon bonded in the unsaturated hydrocarbon compounds. The coupling method supplements the disadvantages of using the conventional organic tin compound in that the byproducts are difficult to be removed and the organic tin compounds are toxic.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A coupling method between intramolecular carbon for preparing a cyclic compound represented by the following chemical formula 1 or 2 having a vinyl group as a substituent by in situ reacting an allyl derivative represented by the following chemical formula 3 or 4 containing an unsaturated hydrocarbon compound with indium (In) or an indium halide and a tertiary anime represented by the following chemical formula 5 to form an allyl-indium compound, wherein Z of the allyl derivative represented by the following chemical formula 3 or 4 is substituted with indium, and carrying out the intramolecular cyclization reaction in situ. 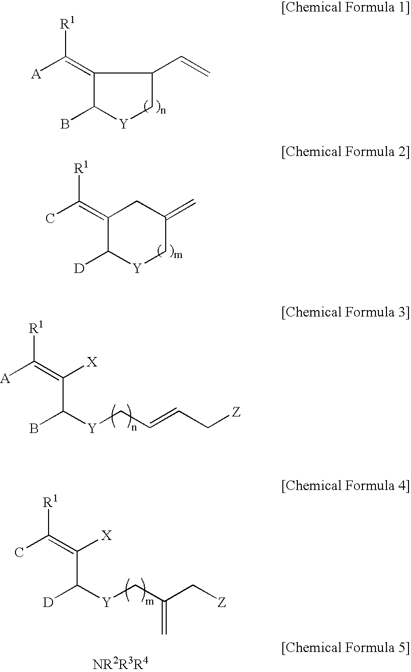
wherein, X is Cl, Br or I; Y is (CH₂)ₚCR¹¹R¹², NR¹³ or (CH₂)_{q}ONR¹⁴; Z is Cl, Br, I, OAc or OCO₂CH₃; A, B, C and D are each independently hydrogen, alkyl having 1 to 5 carbon atoms, phenyl or benzyl, or may form an alkyl ring or fused ring by being bonded to alkylene having 2 to 10 carbon atoms or alkylene having 2 to 10 carbon atoms containing a fused ring; R is each independently hydrogen, fluoro-substituted or -unsubstituted alkyl having 1 to 5 carbon atoms, alkoxy having 1 to 5 carbon atoms, alkylcarbonyl having 1 to 5 carbon atoms or alkoxycarbonyl having 1 to 5 carbon atoms; R², R³ and R⁴ are each independently alkyl having 1 to 5 carbon atoms, phenyl or benzyl; R¹¹ and R¹² are each independently hydrogen alkyl having 1 to 5 carbon atoms, alkoxy having 1 to 5 carbon atoms, alkylcarbonyl having 1 to 5 carbon atoms or alkoxycarbonyl having 1 to 5 carbon atoms; R¹³ is alkyl having 1 to 5 carbon atoms or SO₂R²¹; R¹⁴ is t-butoxycarbonyl or alkyl having 1 to 5 carbon atoms; R²¹ is alkyl having 1 to 5 carbon atoms or phenyl; n is an integer of 1 to 4; m is an integer of 1 to 3; p is an integer of 0 to 5; and q is an integer of 1 to 3.

2. The method according to claim 1, wherein the cyclic compound represented by the chemical formula 1 or 2 having a vinyl group as a substituent is selected from the following compounds.

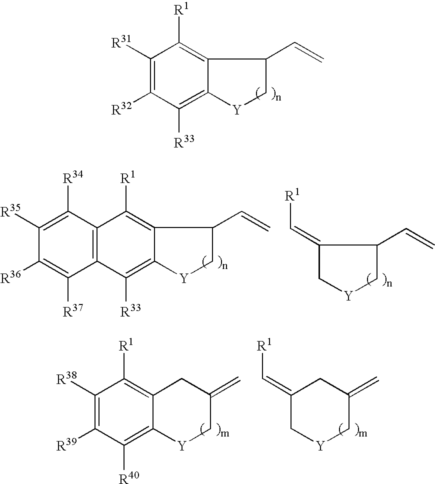

3. The method according to claim 1, wherein the allyl derivative represented by the chemical formula 3 is selected from the following chemical formulas 6 to 8. 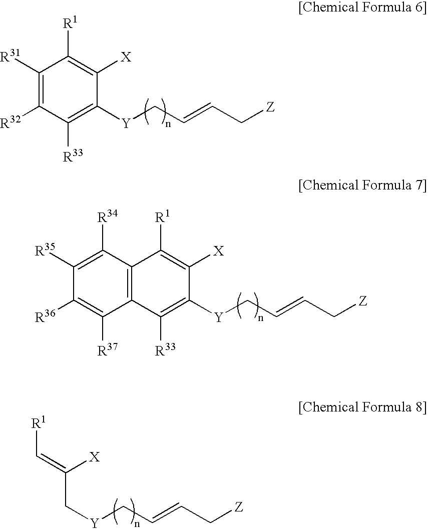
wherein, R¹, X, Y, Z and n are the same as defined in the chemical formula 3, and R³¹ to R³⁷ are each independently hydrogen, alkyl having 1 to 5 carbon atoms, alkoxy having 1 to 5 carbon atoms, alkylcarbonyl having 1 to 5 carbon atoms or alkoxycarbonyl having 1 to 5 carbon atoms.

4. The method according to claim 1, wherein allyl derivative represented by the chemical formula 4 is selected from the following chemical formulas 9 to 10. 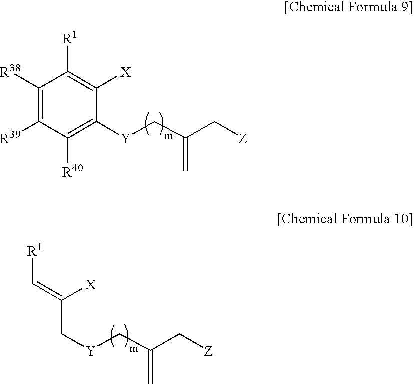
wherein, R¹, X, Y, Z and m are the same as defined in the chemical formula 4, and R³⁸ to R⁴⁰ are each independently hydrogen, alkyl having 1 to 5 carbon atoms, alkoxy having 1 to 5 carbon atoms, alkylcarbonyl having 1 to 5 carbon atoms or alkoxycarbonyl having 1 to 5 carbon atoms.

5. The method according to claim 1, wherein the palladium catalyst is selected from the group consisting of PdCl₂, Pd(OAc)₂, Pd(CH₃CN)₂Cl₂, Pd(PhCN)₂Cl₂, Pd₂dba₃CHCl₃ and Pd(PPh₃)₄.

6. The method according to claim 5, wherein the palladium catalyst is Pd₂dba₃CHCl₃ or Pd(PPh₃)₄.

7. The method according to claim 1, wherein R⁵, R⁶ and R⁷ of the tertiary amine represented by the chemical formula 5 are each independently methyl, ethyl or butyl.

8. The method according to claim 1, wherein the lithium halogen (LiX, wherein X is Cl, Br or I) is used as an additive.

9. The method according to claim 1, wherein the cyclization reaction is carried out in presence of dimethylformamide (DMF) or tetrahydrofuran (THF).
